(19) United States
(12) Patent Application Publication
(10) Pub. No.: US 2004/0102377 A1
(43) Pub. Date: May 27, 2004

(54) Method and composition for prophylaxis of diabetes

(76) Inventors: Irene Perrin, Lausanne (CH); Anthony C. Huggett, Grandvaux (CH); Eduardo Schiffrin, Crissier (CH)

Correspondence Address: BELL, BOYD & LLOYD LLC, P. O. BOX 1135, CHICAGO, IL, 60690-1135, US

(21) Appl. No.: 10/474,560

(22) PCT Filed: Mar. 27, 2002
(86) PCT No.: PCT/EP02/03485

(30) Foreign Application Priority Data
Apr. 10, 2001 (EP) 01107866.4

(51) Int. Cl. A61K 38/18

(52) U.S. Cl. 514/12

(57) ABSTRACT

A nutritional composition for the prophylaxis of insulin-dependent diabetes mellitus type 1 in infants. The nutritional composition contains a protein source, which includes casein rich in active TGF-β2. The casein rich in active TGF-β2 favours tolerance to oral antigens in an infant susceptible to insulin-dependent diabetes mellitus type 1.

### FIELD OF THE INVENTION

This invention relates to a nutritional composition, which may be used in the prophylaxis of infants at risk of diabetes. The invention also relates to methods for the prophylaxis of infants at risk of diabetes, and the use of casein rich in active TGF-β in the nutritional composition.

### BACKGROUND OF THE INVENTION

Insulin-dependent diabetes mellitus type 1 (IDDM) is a T cell-mediated autoimmune disease resulting from the selective destruction of insulin-producing β-cells in the pancreas. It is one of the most frequent chronic diseases of childhood, and the incidence increases rapidly in developed countries, especially among young children. IDDM is associated with serious secondary health complications and a life expectancy reduced by 15 years (Kraine M. R., Tisch R. M. (1999) The role of environmental factors in insulin-dependent diabetes mellitus: an unresolved issue. Environmental Health Perspectives 107(suppl. 5):777-781; EURODIAB ACE Study Group (2000) Variation and trends in incidence of childhood diabetes in Europe. The Lancet 355:873-876).

The development of IDDM is based on the interaction between genetic predisposition and environmental factors. The immune mechanisms leading to β-cell destruction, the nature and mechanisms of action of the environmental factors are largely unknown. Diet is suspected to play an important role as an environmental trigger, whereby attention has focussed mainly on the early introduction of cow milk proteins (CMPs) in infant nutrition. Proposed mechanisms of action include activation of autoreactive T cells by milk proteins showing sequence homology with β-cell autoantigens (bovine serum albumin/ICA69, β-casein/GLUT-2, β-lactoglobulin/retinol-binding protein), crossreaction of immune reponses against bovine insulin with human insulin, release of immunomodulatory β-casomorphin-7 from β-casein variant A1 during digestion. Epidemiological studies in humans and feeding studies with the major animal models of human IDDM, the diabetes-prone BioBreeding (BB) rat and the non-obese diabetic (NOD) mouse, have failed to provide conclusive evidence about the implication of CMPs as environmental triggers of IDDM (Harrison L. C., Honeyman M. C. (1999) Cow's milk and type 1 diabetes. The real debate is about mucosal immune function. Diabetes 48:1501-1507; Norris J. M., Pietropaolo M. (1999) Controversial topics series: milk proteins and diabetes. Journal of Endocrinological Investigation 22:568-580; Schrezenmeir J., Jagla A. (2000) Milk and diabetes. Journal of the American College of Nutrition 19:176S-190S).

It is increasingly acknowledged that IDDM is a complex multifactorial disease. The fundamental defect conferring susceptibility to IDDM is thought to be a genetically determined dysregulation of the gut immune system, resulting in defective tolerance to oral antigens (Harrison L. C., Honeyman M. C. (1999) Cow's milk and type 1 diabetes. The real debate is about mucosal immune function. Diabetes 48:1501-1507; Schrezenmeir J., Jagla A. (2000) Milk and diabetes. Journal of the American College of Nutrition 19:176S-190S; Vaarala O. (1999) Gut and the induction of immune tolerance in type 1 diabetes. Diabetes/Metabolism Research and Reviews 15:353-361). Moreover, in the BB rat model of human IDDM and possibly in human diabetic patients the permeability of the intestinal mucosal barrier is increased, thus facilitating the uptake of luminal antigens and their contact with the immune system (Meddings J. B., Jarand J., Uibanski S. J., Hardin J., Gall D. G. (1999) Increased gastrointestinal permeability is an early lesion in the spontaneously diabetic BB rat. American Journal of Physiology 39:G951-G957; Carratù R., Secondulfo M., de Magistris L., Iafusco D., Urio A., Carbone M. G., Pontoni G., Carteni M., Prisco F. (1999) Altered intestinal permeability to mannitol in diabetes mellitus type 1. Journal of Pediatric Gastroenterology 28:264-269). Therefore, multiple different environmental factors are likely to contribute to disease development. The risk components may differ between susceptible individuals, depending on their genetic set-up (Dahlquist G. (1998) The aetiology of type 1 diabetes: an epidemiological perspective. Acta Paediatrica 87(suppl 425):5-10). During years, attempts at IDDM prevention have included the search for identification and elimination of key diabetogens. In view of the current state of knowledge about IDDM pathogenesis, this does no longer appear as a promising approach for disease prevention.

As a conclusion, there is no possibility at the moment to prevent IDDM by nutritional means. Nor is there a food component known to be able to improve the development of tolerance to oral antigens in the gut immune system. This mechanism is thought to be defective in subjects susceptible to IDDM.

Transforming growth factor-β (TGF-β) is a multifunctional cytokine with important immunomodulatory properties which is present in many tissues and body fluids including milk. The isolation of TGF-β from milk is considered to be an expensive and labour-intensive process, described in EP 0313515 (Ciba-Geigy). Recombinant TGF-β is available, however besides its high costs it is a therapeutic agent and therefore not suitable for food application. Therefore, for various reasons there is no possibility at the moment to add pure TGF-β to food products. On the other hand, the known methods of industrial purification of milk components (for example the production of a powder of casein for nutritional formulas) are supposed to destroy TGF-β in a way that it doesn't retain its activity if consumed. According to the state of the art, TGF-β is thought not to be present or to be irreversibly inactivated in nutritional formulas (e.g. milk-based infant formulas) on the market as of to date. Last but not least, the role of TGF-α in the multifactorial pathogenesis of IDDM is not yet clarified, although there is speculation that this cytokine might play a role in the protection against autoimmune diseases (Prud'homme G. J., Piccirllo C. A. (2000) The inhibitory effects of transforming growth factor-beta 1 in autoimune diseases. Journal of Autoinmnunity 14:23-42).

The present invention addresses these problems.

### SUMMARY OF THE INVENTION

Accordingly, in a first aspect, this invention provides a nutritional composition for the prophylaxis of insulin-dependent diabetes mellitus type 1 in susceptible infants, which comprises a protein source including casein rich in TGF-β2.

Furthermore, the invention also provides a nutritional composition for the promotion of tolerance to oral antigens in an infant susceptible to insulin-dependent diabetes mellitus type 1, which comprises a protein source including casein rich in TGF-β2.

This invention also provides the use of casein rich in active TGF-β2 in the preparation of a nutritional composition for the prophylaxis of insulin-dependent diabetes mellitus type 1 in infants.

This invention then provides the use of casein rich in active TGF-β2 in the preparation of a nutritional composition promoting tolerance to oral antigens in an infant susceptible to insulin-dependent diabetes mellitus type 1.

In a next aspect, the invention provides a method for the prophylaxis of insulin-dependent diabetes mellitus type 1 in susceptible infants which comprises administering to infants a nutritional composition having a protein source including casein rich in TGF-β2.

In another aspect, the invention provides a method for the promotion of tolerance to oral antigens in an infant susceptible to insulin-dependent diabetes mellitus type 1, which comprises administering to the infant a nutritional composition having a protein source including casein rich in TGF-β2.

### DETAILED DESCRIPTION OF THE PREFERRED EMBODIMENTS

This invention is based on the surprising discovery that TGF-β2 (the isoform of TGF-β which predominates in milk) is not destroyed in all known processes for the industrial manufacture of casein. TGF-β2 retains its ability to be active in the digestive tract in certain “mild” or sparing processes for the production of casein. Hence, in most nutritional formulas comprising casein and TGF-β2, the cytokine has lost its ability to exert an immunomodulatory activity in the gastrointestinal tract. However, surprisingly, there are known procedures for the production of casein from skimmed milk which preserve TGF-β2 in an active form, especially those that use decationised whey for the precipitation of the casein. An example is given in FR 1'469'793.

This invention is also based upon the discovery that a nutritional composition which comprises a protein source including casein rich in active TGF-β2, reduces the IDDM frequency in a susceptible animal model. Furthermore it has been found that a nutritional composition which comprises a protein source including casein rich in active TGF-β2, modifies the immune microenvironment in the gut to a pattern that is favourable for the devlopment of tolerance to oral antigens and that it reduces gut permeability. These mechanisms may account for the IDDM-preventive effect.

It has thus surprisingly been found that the immunomodulatory cytokine TGF-β2, if present in food in an active form, is able to promote tolerance to oral antigens and thereby to reduce IDDM frequency.

Furthermore, it has surprisingly been found that casein rich in active TGF-β2 is effective in preventing IDDM by promoting tolerance to oral antigens.

Therefore, feeding a formula rich in active TGF-β to infants and children susceptible to IDDM may inhibit pathogenic mechanisms leading to the development of IDDM.

For the purpose of the present invention the term “active TGF-β” is intended to include TGF-β that is not irreversibly inactivated. Hence, the term includes TGF-β that is not active, but that may be activated by passage through the digestive tract, for example.

In a preferred embodiment of the nutritional compositions, the uses or the methods according to the present invention, the TGF-β2 may be present in an active form or in a form that is activated during passage through the digestive tract.

In a preferred embodiment of the nutritional compositions, the uses or the methods according to the present invention, the composition for the prophylaxis of insulin dependent diabetes mellitus type 1 may contain 0,5 μg to 5 μg, TGF-β2 per g of casein. Preferably, 1 μg to 3,5 μg, more preferably 1.2 μg to 2.0 μg, in particular 1.6 μg of TGF-β2 per g of casein may be used.

In a preferred embodiment of the nutritional compositions, the uses or the methods according to the present invention, the casein rich in TGF-β2 may be used as a main protein source of the nutritional composition, for example.

The casein may be provided in free form or in the form of a salt; for example, a sodium salt. It is also possible to provide the casein as a calcium or sodium-calcium salt.

The casein rich in TGF-β2 may not be produced by any process known in the art. It is important that the casein is obtained in a “mild” way, for example as illustrated in FR 1'469'793. This document discloses a process for obtaining casein, with a concurrent production of whey for the manufacture of lactose and nutritional compositions for mast, by precipitating casein by lowering the pH, said process comprising, solely or in combination with each other, the following features:

1. Decreasing the pH of skimmed milk by addition of whey, the pH of which has been decreased by exchange of cations;

2. The whey used as precipitation agent having been liberated at least partially from lactose before the treatment of exchange of cations;

3. The whey used as precipitation agent having been liberated at least partially from albumin before the treatment of exchange of cations;

4. The whey used as precipitation agent having been liberated from albumin and from lactose together;

5. The whey used as precipitation agent being obtained by diluting the slurry of whey after removal of lactose in view of raising its pH to 4.3 to 4.8, with the aid of a product like sweet whey of cheese or the water after rinsing the lactose, then heating (the diluted whey) to 90 to 95° C., and, after having eliminated the albumin in so doing, treating it in a cation-exchanger.

If desired, the protein source may include different types of hydrolysed protein; for example egg white protein, soy protein, rice protein, pea protein and the like.

The protein preferably provides about 5% to about 30% of the energy of the nutritional composition; for example about 10% to about 20% of the energy. When used as an infant formula, the nutritional composition preferably contains about 1.8 g/100 kcal to about 3 g/100 kcal of the protein source. The remaining energy of the nutritional composition may be provided in the form of carbohydrates and fats.

If the nutritional composition includes a fat source, the fat source preferably provides about 5% to about 55% of the energy of the nutritional composition; for example about 20% to about 50%, or 25% to about 35% of the energy. The lipids making up the fat source may be any suitable fat or fat mixture. Vegetable fats are particularly suitable; for example soy oil, palm oil, coconut oil, safflower oil, sunflower oil, corn oil, canola oil, lecithins, and the like. Animal fats such as milk fats may also be added if desired.

If the nutritional composition includes a carbohydrate source, the carbohydrate source preferably provides about 40% to about 80% of the energy of the nutritional composition. For example, 45% to 54% or 61% to 75% may be used. Any suitable carbohydrates may be used, for example sucrose, lactose, maltose, glucose, corn syrup solids, pre-cooked or gelatinised starch, and maltodextrins, and mixtures thereof. If desired, the carbohydrate source may be free, or substantially free, of lactose.

Suitable vitamins, trace elements and minerals are included in the usual manner to meet the appropriate guidelines in the various countries.

The energy density of the nutritional composition is preferably about 50 kcal/100 ml to about 90 kcal/100 ml. However, the energy density may also be higher that 110 kcal/100 ml, for example 105 to 130 kcal/100 ml.

The nutritional composition may be prepared in any suitable manner. For example, for a nutritional composition intended as a complete diet, the nutritional formula may be prepared by blending together the protein source, the carbohydrate source, and the fat source in appropriate proportions. If used, the emulsifiers may be included in the blend. The vitamins and minerals may be added at this point but are usually added later to avoid thermal degradation. Any lipophilic vitamins, emulsifiers and-the like may be dissolved into the fat source prior to blending. Water, preferably water, which has been subjected to reverse osmosis, may then be mixed in to forma liquid-mixture.

The liquid mixture may then be thermally treated to reduce bacterial loads. For example, the liquid mixture may be rapidly heated to a temperature in the range of about 80° C. to about 110° C. for about 5 seconds to about 5 minutes. This may be carried out by steam injection or by heat exchanger; for example a plate heat exchanger.

The liquid mixture may then be cooled to about 60° C. to about 85° C.; for example by flash cooling. The liquid mixture is then homogenised; for example in two stages at about 7 MPa to about 40 MPa in the first stage and about 2 MPa to about 14 MPa in the second stage. The homogenised mixture may then be further cooled to add any heat sensitive components; such as vitamins and minerals. The pH and solids content of the homogenised mixture is conveniently standardised at this point.

If it is desired to produce a powdered nutritional composition, the homogenised mixture is transferred to a suitable drying apparatus such as a spray drier or freeze drier and converted to powder. The powder should have a moisture content of less than about 5% by weight.

If it is desired to produce a liquid nutritional composition, the homogenised mixture is preferably aseptically filled into suitable containers. Suitable apparatus for carrying out aseptic filing of this nature is commercially available. The liquid nutritional composition may be in the form of a ready to feed formula having a solids content of about 10 to about 14% by weight or may be in the form of a concentrate; usually of solids content of about 20 to about 26% by weight.

The nutritional composition may also be in other suitable forms; for example an infant cereal.

The nutritional compositions are preferably in the form of a complete diet such that, when used as the sole source of nutrition, essentially all daily energy, nitrogen, lipid, vitamin, mineral and trace elements. However, the nutritional composition may also be in the form of a supplement. The nutritional composition may be used as an infant formula or as a follow-up formula.

The nutritional compositions may be used as a nutritional support for infants at risk of IDDM. As such, the nutritional composition maybe used as a prophylactic for infants susceptible to IDDM. The amount of the nutritional composition required to be fed to the infant will vary depending upon factors such as the infant's condition, the infant's body weight, the age of the infant, and whether the nutritional composition is the sole source of nutrition. In general, sufficient of the nutritional composition is administered to provide the infant with about 1 g protein to about 4.0 g protein per kg of body weight per day. If the nutritional composition is used as a supplement to other foods, the amount of the nutritional composition that is administered daily may be decreased accordingly.

The invention is now further explained with reference to specific examples.

### EXAMPLE 1

A powdered infant formula is prepared from casein which contains about 1.6 μg of TGF-β2 per g of casein, palm olein, coconut oil, safflower oil, lecithin, maltodextrin, lactose, vitamins and minerals in a conventional manner. The infant formula contains about 30 μg of TGF-β2 per 100 g of the formula, on a dry basis. The formula is in accordance with EC directives 91/321/EEC and 96/4/EC.

### EXAMPLE 2

The diabetes-prone BioBreeding (BB) rat is used. It is one of the best characterised animal models for human IDDM, close to the human disease in many aspects (spontaneous development of IDDM, interaction of genetic and environmental factors, auto-immune mechanisms involved, equal frequency in both sexes, peak onset around puberty, clinical symptoms and pancreas histology).

Groups of 40 BB rats (20 per sex) obtained from Animal Resources Division, Health Canada, are weaned onto the experimental diets, which are already fed to the respective groups of dams during pregnancy and lactation. All diets are based on AIN-93G, a nutritionally balanced semi-synthetic standard rodent diet, but with the protein source (18%) adapted as follows:

Group A: Whey protein concentrate (WPC; positive control)

Group B: Extensively hydrolysed casein (negative control)

Group C: Acid casein rich in TGFβ2

Group D: K-caseinate, devoid of TGFβ2

Group E: WPEC/Acid casein rich in TGFβ2 at 1:1

Group F: WPC/K-caseinate, devoid of TGFβ2 at 1:1

The experimental animals are kept under SPF conditions and have free access to food and drinking water. Body weight, food and water consumption and urinary glucose are recorded regularly. IDDM is diagnosed based on the blood glucose level (>11.1 mmol/l) and glucosuria. Diabetic animals are immediately sacrified and the diagnosis is verified histologically. The experiment is terminated with the sacrifice of all surviving animals at the age of 5 months. Blood and urinary glucose is recorded and the pancreas is examined histologically.

At the age of 70 days, gut permeability is analysed in 20 (10 per sex) prediabetic rats per group. The animals are fasted and placed in a metabolic cage overnight. In the morning the urine is collected for base-line measurement. The animals then receive by gavage 1 ml of a solution containing lactulose/mannitol. After a further fasting period of 6 hours the urine is collected. Gut permeability is expressed as the proportion of lactulose/mannitol excretion in the urine.

At the age of 70 days, additional subgroups of 20 (10 per sex) prediabetic rats per group are sacrified for the analysis of cytokines in Peyer's patches (gut-associated lymphoid tissue) and pancreas. Cytokines analysed are IFN-γ (Th1), IL-10 (Th2) and TGF-β (Th3), as well as INOS (marker of destructive inflammation).

The results after 5 months are given in Table 1:

TABLE 1
Influence of dietary protein source on IDDM incidence,
gut permeability and tissue cytokine profiles in
diabetes-prone BB rats.

| | | | | Cytokines |
|---|---|---|---|---|
| | | IDDM | Gut | (gut, |
| Group | Protein source | incidence | permeability | pancreas) |
| A | WPC | High | High | Th1 |
| B | Hydrolysed casein | Low | High | Th2/Th3 |
| C | Casein + TGF-β2 | Low | Decreased | TH2/Th3 |
| D | Casein − TGF-β2 | Moderate | High | Th1 |
| E | WCP/Casein + TGF- | Low- | Decreased | Th2/Th3 |
| | β2 | moderate | | |
| F | WPC/Casein − TGF- | Moderate- | High | Th1 |
| | β2 | high | | |

Table 1 shows that a diet based on extensively hydrolysed casein (B) which is almost devoid of immunoreactive peptides is clearly less diabetogenic than a diet based on intact protein (C). The table also shows that an intact casein rich in active TGF-β2 (C) tends to be less diabetogenic than an intact casein very poor in active TGF-β2 (D). If these caseins are mixed with a strongly diabetogenic protein such as WPC (E and F), the IDDM-reducing effect is more prominent if active TGF-β2 is present in the casein (E). These findings indicate that the IDDM-preventive effect observed is mainly due to the preservation of active TGF-β2 by a particular processing for the manufacture of casein.

Table 1 also shows that gut permeability expressed as the lactulose:mannitol ration excreted in the urine was lower in those groups fed diets containing active TGF-β2.

Table 1 also demonstrates that a diabetogenic diet (A) is associated with a Th1 cytokine pattern in gut immune system and pancreas which reflects the destructive immune reaction thought to mediate IDDM. In contrast a protective diet (B) is associated with a TH2/Th3 cytokine pattern which reflects an immune response suppressing autoimmune destruction (Scott F. W., Cloutier H. E., Kleemann R. et al (1997) Potential mechanisms by which certain foods promote or inhibit the development of spontaneous diabetes in BB rats. Diabetes 46:589-598). Diets containing active TGF-β2 (C,E) induced a shift from a Th1 to a Th2/Th3 pattern when compared to the identical diets poor in TGF-β2 (D,F.). A Th2/Th3 cytokine pattern in the gut immune system is thought to mediate tolerance to oral antigens (Weiner H. L. (1997) Oral tolerance: immune mechanisms and treatment of autoimmune diseases. Immunology today:18:335-343).

In conclusion, our experiment indicates that a dietary protein source which contains active TGF-β2 reduces IDDM frequency in a susceptible animal model, decreases gastrointestinal permeability and induces an immune microenvironment in the gut which is favourable to the development of tolerance to oral antigens.

1. A nutritional composition for the prophylaxis of insulin-dependent diabetes mellitus type 1 in infants, which comprises a protein source including casein rich in TGF-β2.

2. A nutritional composition for promoting the induction of tolerance to oral antigens in an infant susceptible to insulin dependent diabetes mellitus type 1, which comprises a protein source including casein rich in TGF-β2.

3. Composition according to claim 1 or 2, wherein the casein rich in TGF-β2 is used as a main protein source of the nutritional composition.

4. Composition according to any of claim 1 to 3, wherein the TGF-β2 is present in an active form or in a form that is activated during passage through the digestive tract.

5. Composition according to any of claim 1 to 4, wherein the casein contains 0.5 μg to 5 μg TGF-β2 per g of casein.

6. The use of casein rich in TGF-β2 in the preparation of a nutritional composition for the prophylaxis of insulin-dependent diabetes mellitus type 1 in infants.

7. The use of casein rich in TGF-β2 in the preparation of a nutritional composition promoting the induction of tolerance to oral antigens in an infant susceptible to insulin-dependent diabetes mellitus type 1.

8. Use according to claim 6 or 7, wherein the casein rich in TGF-β2 is used as a main protein source of the nutritional composition.

9. Use according to any of claim 6 to 8, wherein the TGF-β2 is present in an active form or in a form that is activated during passage through the digestive tract.

10. Use according to any of claim 6 to 9, wherein the casein contains 0.5 μg to 5 μg TGF-β2 per g of casein.

11. Method for the prophylaxis of insulin-dependent diabetes mellitus type 1 in infants, which comprises administering to infants a nutritional composition having a protein source including casein rich in TGF-β2.

12. Method for promoting the induction of tolerance to oral antigens in an infant susceptible to insulin-dependent diabetes mellitus type 1, which comprises administering to the infant a nutritional composition having a protein source including casein rich in TGF-β2.
